(19) United States
(12) Patent Application Publication
(10) Pub. No.: US 2026/0367026 A1
(43) Pub. Date: Nov. 19, 2026

(54) GRAPHENE-BASED CHEMICAL SENSOR SYSTEM

(71) Applicants: Eric Nallon, Earlysville, VA (US); Maurice Jackson, COLORADO SPRINGS, CO (US); Baabi Das, Arlington, VA (US)

(72) Inventors: Eric Nallon, Earlysville, VA (US); Maurice Jackson, COLORADO SPRINGS, CO (US); Baabi Das, Arlington, VA (US)

(73) Assignee: Zansors LLC, Arlington, VA (US)

(21) Appl. No.: 19/142,807

(22) PCT Filed: Dec. 29, 2023
(86) PCT No.: PCT/US2023/086357

§ 371 Date: Jun. 24, 2025

Related U.S. Application Data
(60) Provisional application No. 63/436,160, filed on Dec. 30, 2022.

(51) Int. Cl. G01N 27/12; G16C 20/20; G16C 20/70

(52) U.S. Cl. CPC G01N 27/125; G16C 20/20; G16C 20/70

(57) ABSTRACT

The present invention discloses a graphene-based chemical sensor system. The system comprises a sensor module comprising a substrate, a plurality of pairs of electrodes disposed over the substrate, and a sensing layer disposed over each pair of electrodes on the substrate to form an array of sensing surfaces. The sensing layer is an integrated surface functionalization mask having an opening well for each sensing surface. Each opening well comprises a different polymer material. The surface functionalization mask enables simultaneous deposition of various polymer materials to the sensing surfaces using microarray printing. The sensor module, on presenting chemical compounds on the sensing surfaces and applying constant bias across the electrodes, is configured to create a sensor data with cross-reactive response characteristics due to temporary time-dependent change in resistance of the graphene. The substrate further comprises silicon (Si), thermally grown silicon dioxide (SiO₂) and graphene. The electrode comprises titanium/gold (Ti/Au) interdigitated electrode.

### CROSS-REFERENCE TO RELATED APPLICATIONS

This application claims priority to PCT International application No. PCT/US2023/086357, filed on Dec. 29, 2023, which further claims the priority of U.S. Provisional Patent Application 63/436,160, filed on Dec. 30, 2022, the entire contents of which are incorporated herein by reference.

### TECHNICAL FIELD

The present invention generally relates graphene-based sensor. More specifically, the present invention relates to a graphene-based chemical sensor system configured to provide a broad responding behavior to a variety of chemical compounds.

### BACKGROUND

As chemical and biological sensors continue to be integrated into our everyday lives, the demand for a highly dimensional, adaptable sensor array becomes increasingly important. The chemical sensor is an analyzer that responds to a particular analyte selectively and reversibly way and transforms input chemical quantity, ranging from the concentration of a specific sample component to a total composition analysis, into an analytically electrical signal.

Individual electronic chemical sensors may be designed to be specific for a single target chemical, broadly responsive to a class of chemicals, or have enhanced sensitivity for particular chemical interactions. Creating a chemically differentiated sensor array is more complex than the creation of a single sensor. Graphene-based chemical sensors offer the possibility of ultrahigh sensitivity detection of chemical compounds in mixtures with air at room temperature and atmospheric pressure.

Graphene is a single-layer, 2D material. Graphene comprises carbon atoms arranged in a hexagonal lattice, with extraordinary electrical, mechanical, thermal, and optical properties due to its 2D, sp2 bonded structure. The 2D structure provides more potential to graphene as a chemical sensing material by entirely exposing its surface to the surrounding environment. In this configuration, every carbon atom in graphene is a surface atom, providing the greatest possible surface area per unit volume, so that electron transport is highly sensitive to adsorbed molecular species. Graphene has gained much attention since its discovery in 2004, but has not been realized in many commercial electronics. Graphene has the potential to be a revolutionary material for use in chemical sensors due to its excellent conductivity, large surface area, low noise, and versatile surface for functionalization. However, prior works have not addressed the ability to apply multiple surface modifications on a single graphene chip, which is addressed in this invention.

Therefore, there is a need for a graphene-based chemical sensor system configured to provide a broad responding behavior to a variety of chemical compounds. Also, there is a need for a graphene-based chemical sensor to provide increased sensitivity and selectivity.

### SUMMARY OF THE INVENTION

The present invention discloses a graphene-based chemical sensor system. The system comprises at least one sensor module, a data logger module in communication with the sensor module and a computing device in communication with the data logger module. The sensor module comprises a substrate, a plurality of pairs of electrodes disposed over the substrate, and a sensing layer disposed over each pair of electrodes on the substrate to form an array of sensing surfaces. The sensing layer is an integrated surface functionalization mask having an opening well for each sensing surface. Each opening well comprises a different polymer material. The surface functionalization mask enables simultaneous deposition of various polymer materials to the sensing surfaces using microarray printing.

The sensor module, on presenting chemical compounds on the sensing surfaces and applying constant bias across the electrodes, is configured to create a sensor data with cross-reactive response characteristics due to temporary time-dependent change in resistance of the graphene. In one embodiment, the substrate comprises silicon (Si), thermally grown silicon dioxide (SiO₂), and graphene. In one embodiment, the electrode is a titanium/gold (Ti/Au) interdigitated electrode. In one embodiment, the sensing layer is an epoxy-based negative photoresist. In one embodiment, the sensing layer is a SU8 layer. The sensor module further comprises at least one humidity sensor and at least one temperature sensor.

The data logger module is configured to collect sensor data from the sensor module. The data logger module comprises a start and stop button to manually control logging of the sensor data. The computing device comprises an artificial intelligence module. The artificial intelligence module is configured to classify and identify a predefined set of chemical compounds. The computing device is configured to analyze the sensor data to identify chemical compounds by measuring a time-dependent change in resistance of the sensing surfaces. The analysis of the sensor data involves preprocessing the sensor data, extracting features from the sensor data, reducing the dimensionality of the established features, if necessary, and classifying chemical compounds.

The computing device is further configured to re-train the artificial intelligence module to identify a set of new chemical compounds and add the new chemical compounds to the predefined set of chemical compounds. The computing device further enables to send commands to the data logger module to manually control logging of the sensor data. The computing device is further configured to plot the cross-reactive response characteristics and monitor sensor module in real time.

In another embodiment, the present invention discloses a graphene-based chemical sensor system. The system comprises a sensor module comprising a substrate, a plurality of pairs of electrodes disposed over the substrate, and a sensing layer disposed over each pair of electrodes on the substrate to form an array of sensing surfaces. The sensing layer is an integrated surface functionalization mask having an opening well for each sensing surface. Each opening well comprises a different polymer material. The surface functionalization mask enables simultaneous deposition of various polymer materials to the sensing surfaces using microarray printing. The sensor module, on presenting chemical compounds on the sensing surfaces and applying constant bias across the electrodes, is configured to create a sensor data with cross-reactive response characteristics due to temporary time-dependent change in resistance of the graphene. In one embodiment, the substrate comprises silicon (Si), thermally grown silicon dioxide (SiO₂), and graphene. In one embodiment, the electrode is a titanium/gold (Ti/Au) interdigitated electrode. In one embodiment, the sensing layer is an epoxy-based negative photoresist. In one embodiment, the sensing layer is a SU8 layer.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 shows a graphene sensor array in one embodiment of the present invention.

FIG. 2A shows a photo-lithography metallization mask design in one embodiment of the present invention.

FIG. 2B shows a photo-lithography integrated surface functionalization mask in one embodiment of the present invention.

FIG. 3A shows a single sensor of the graphene sensor array in one embodiment of the present invention.

FIG. 3B shows a graphene substrate in one embodiment of the present invention.

FIG. 4 shows a graph of a typical time-dependent change in the sensor resistance in one embodiment of the present invention.

FIG. 5 shows a scanning electron microscope image of polymer layers deposited over the graphene sensor electrode array in one embodiment of the present invention.

FIGS. 6A-6C show various microscope images of the graphene sensor in one embodiment of the present invention.

FIG. 6D shows a step profile with a profilometer detailing the various heights of the layers in one embodiment of the present invention.

FIG. 7 shows a method of fabrication of the graphene sensor array in one embodiment of the present invention.

FIG. 8 exemplarily illustrates an environment of a graphene-based chemical sensor system, according to an embodiment of the present invention.

FIG. 9 exemplarily illustrates a chamber board with a sensor module, according to an embodiment of the present invention.

FIG. 10 shows a graph illustrating a typical sensor response in one embodiment of the present invention.

FIG. 11 shows a flowchart of a method for classifying target compounds in one embodiment of the present invention.

FIGS. 12A-12C show the change in resistance of various sensors in one embodiment of the present invention.

FIG. 13 shows a correlation heatmap between any two modified sensors in one embodiment of the present invention.

FIG. 14 shows algorithmic steps to determine an optimal set of polymer coatings in one embodiment of the present invention.

FIG. 15 shows a graph illustrating a diverse sensor response against a broad range of chemicals and compounds in one embodiment of the present invention.

FIG. 16 shows a graph illustrating a classification accuracy of the complex odor from multiple scotch whiskies achieved by a single graphene sensor in one embodiment of the present invention.

FIG. 17 shows a graph illustrating a classification accuracy of the complex odor from multiple essential oils achieved by four modified graphene sensors in one embodiment of the present invention.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

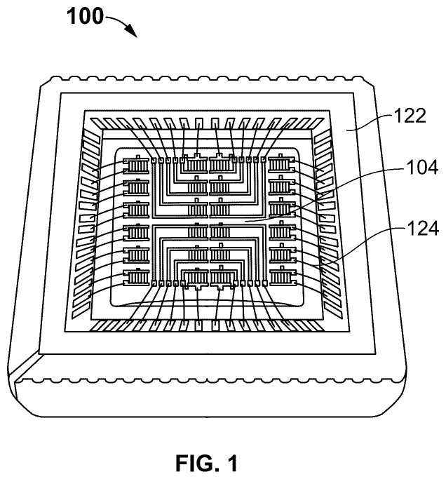

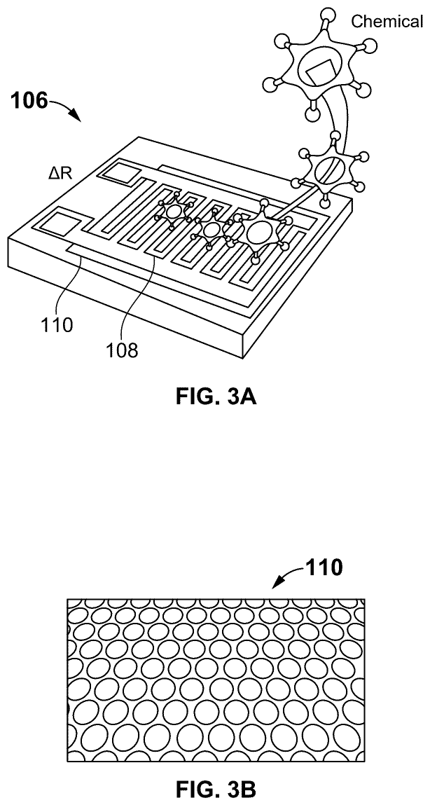

FIG. 1 shows a graphene-based sensor array 100 (also referred as a sensor module 100), according to one embodiment of the present invention. The sensor module 100 is a cross-reactive, graphene-based chemical sensor array. The sensor module 100 comprises a substrate 110 (shown in FIG. 3A), a plurality of pairs of electrodes 108 (shown in FIG. 3A) disposed over the substrate 110 and a sensing layer 104 disposed over each pair of electrodes 108 on the substrate 110 to form an array of sensing surfaces. The sensing layer 104 is an integrated surface functionalization mask having an opening well for each sensing surface. Each opening well comprises a different polymer material. In one embodiment, the substrate 110 comprises silicon (Si), thermally grown silicon dioxide (SiO₂) and graphene. In one embodiment, the electrode 108 is a titanium/gold (Ti/Au) interdigitated electrode. In one embodiment, the sensing layer 104 is an epoxy-based negative photoresist layer. In another embodiment, the sensing layer 104 is a SU8 layer. Further, on presenting chemical compounds on the sensing surfaces and applying constant bias across the electrodes 108, a resistance of the graphene is temporarily modified, resulting in a time-dependent change in resistance. The sensor module 100 is configured to create a sensor data with cross-reactive response characteristics due to temporary time-dependent change in resistance of the graphene.

The sensor module 100 is a resistive sensor array or a sensor network. The sensor module 100 is capable of detecting and classifying a broad range of chemicals, compounds, and complex odors. In one embodiment, the sensor module 100 is designed using a combination of engineering, material science, and machine learning to make it to be adaptable to new targets in the field by means of algorithm updates opposed to hardware changes. In one embodiment, a standard semiconductor processing techniques are used to create the sensor module 100.

The sensor module 100 is fabricated and its performance is evaluated against single compounds and complex odors. The sensor module 100 is created on a single substrate. Further, various polymer coatings applied to each individual sensing surface create a sensor output with cross-reactive response characteristics The sensor module 100 is designed to have a broad responding behavior to a variety of chemical compounds due to the surface modifications applied to the graphene surface.

The sensor module 100, characterized by its small size, low power consumption, resilience, and adaptability, positions it as a viable choice for a remote, autonomously operated sensor. It is deployable across various platforms and designed to fit into a package size similar to that of a tennis ball or hockey puck. The sensor module 100 uses graphene material, which is the heart of the sensor module 100. Graphene possesses properties that are advantageous for chemical sensing and processing chip-based sensors on the micro and nano size at scale. The carbon composition of graphene and the availability of diverse carbon chemistries provide a platform for surface modification. In this manner, individual graphene sensors may be modified with organic groups, metal nanoparticles, enzymes, biomolecules, or polymers and acquire their properties and characteristics.

In one embodiment, the surface modifications such as polymer coatings, of the sensor module 100 provides chemical diversity configured to provide information rich, sorption-based changes in each sensor response. It also provides an electrical modification to the graphene, resulting in substantial enhancements in the sensing response and often a complete reversal in the resistance change direction. This technique extends the inherent, broad selective nature of graphene as the chemical sensing material.

In one embodiment, a variety of single volatile organic compounds and complex odors compounds are identified with very high accuracy using only a portion of the complete set of sensing elements, suggesting that the array contains much more potential for the identification of additional and more complex compounds. The sensor module 100 also operates at room temperature and does not require regenerative techniques such as heat or UV light, which are commonly found in other chemical sensing designs. These results demonstrate for the first time, a cross-reactive graphene chemical sensor array, applicable to compact, low-cost, low-power, and adaptable sensing needs for electronic nose systems.

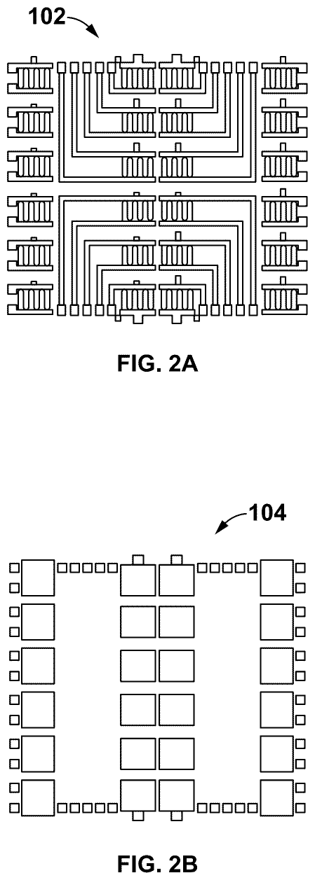

FIGS. 2A-2B shows a 24-element graphene sensor array, according to one embodiment of the present invention. FIG. 2A exemplarily illustrates a photo-lithography metallization mask design 102, according to one embodiment of the present invention. FIG. 2B exemplarily illustrates a photo-lithography integrated surface functionalization mask or sensing layer 104, according to one embodiment of the present invention. In one embodiment, a polymer hard mask is created using standard photo-lithography techniques which forms an integrated surface functionalization mask 104 allowing simultaneous deposition of multiple polymers or other materials to the sensor surface using microarray printing.

FIG. 3A shows a single sensor or a single sensing surface 106 of the sensor module 100, according to one embodiment of the present invention. In one embodiment, the sensor 106 is a chemiresistor device comprised of a pair of electrodes 108. The pair of electrodes 108 is disposed on a chemical substrate 110. In one embodiment, the electrodes 108 is an interdigitated electrodes (IDE) with a specified length, width, and separation. In one embodiment, the substrate 110 may be silicon (Si) or silicon dioxide (SiO₂) or graphene substrate 110 as shown in FIG. 3B. In one embodiment, the sensor 106 has a change in resistance with respect to time when exposed to a chemical compound.

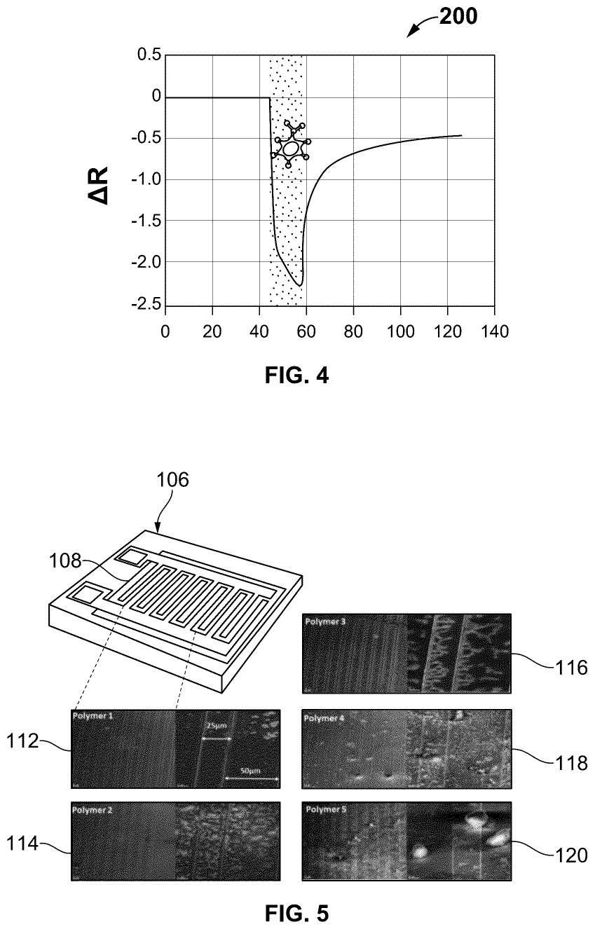

In one embodiment, the sensor resistance is established due to the separation of the electrode fingers and resistive properties of the substrate 110. To operate the sensor 106, a constant bias is applied across the electrodes and the resulting current is measured. When a chemical compound is presented to the sensor surface, the resistance of the graphene substrate 110 is temporarily modified, resulting in a time-dependent change in resistance. FIG. 4 exemplarily illustrates a graph 200 showing the typical time-dependent change in the sensor resistance, according to one embodiment of the present invention. In one embodiment, the sensor resistance varies over time while exposing to a chemical compound. Upon removal of the compound from the sensor 106, the resistance begins to return to its baseline value.

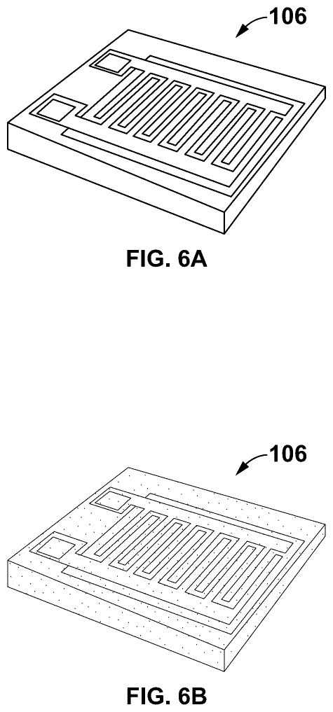

FIG. 5 shows a scanning electron microscope image of a plurality of polymer layers of the sensor 106, according to one embodiment of the present invention. In one embodiment, the sensor 106 comprises five polymer layers (112, 114, 116, 118, and 120). In one embodiment, the polymer layers (112, 114, 116, 118, and 120) are deposited over the electrodes 108 of the sensor 106.

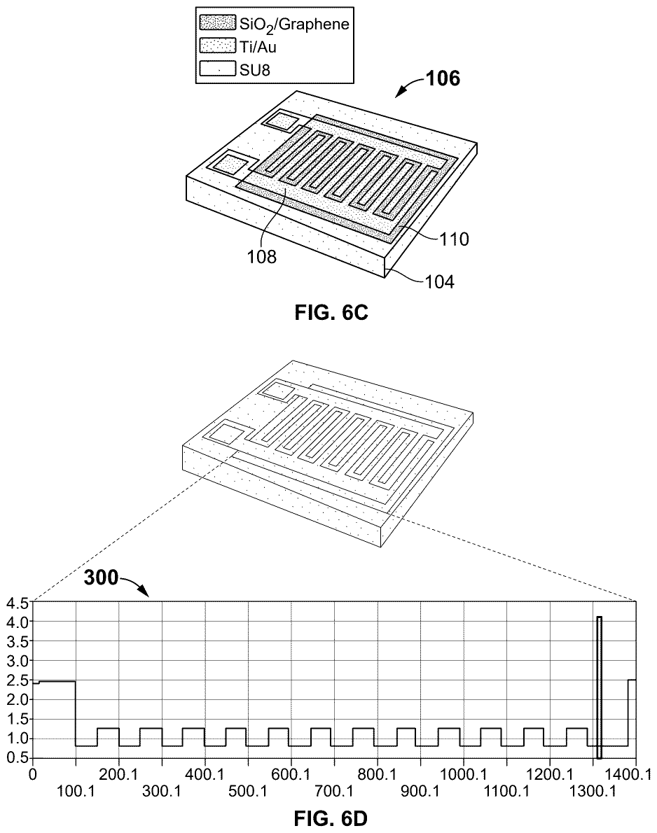

FIGS. 6A-6C shows various microscope images of the graphene sensor 106, according to one embodiment of the present invention. FIG. 6A exemplarily illustrates a color image of the graphene sensor 106. FIG. 6B exemplarily illustrates an intensity image of the graphene sensor 106. FIG. 6C exemplarily illustrates a false height image of the graphene sensor 106. The graphene sensor 106 includes one or more layers with varying heights. In one embodiment, the graphene sensor 106 includes a substrate 110. The substrate 110 may be SiO₂ or graphene substrate. In one embodiment, each graphene sensor 106 includes pair of electrodes 108. The sensor 106 further includes a sensing layer 104. The sensing layer 104 includes a high contrast, epoxy-based photoresist or SU-8. The electrodes 108 defines a metal layer. The metal layer may be Ti/Au layer. The metal layer is deposited over the substrate 110.

Referring to FIG. 6D, a step profile 300 with a profilometer detailing the various heights of the layers, according to one embodiment of the present invention. In one embodiment, the step profile 300 demonstrates starting and ending of the SU-8 polymer integrated surface functionalization mask.

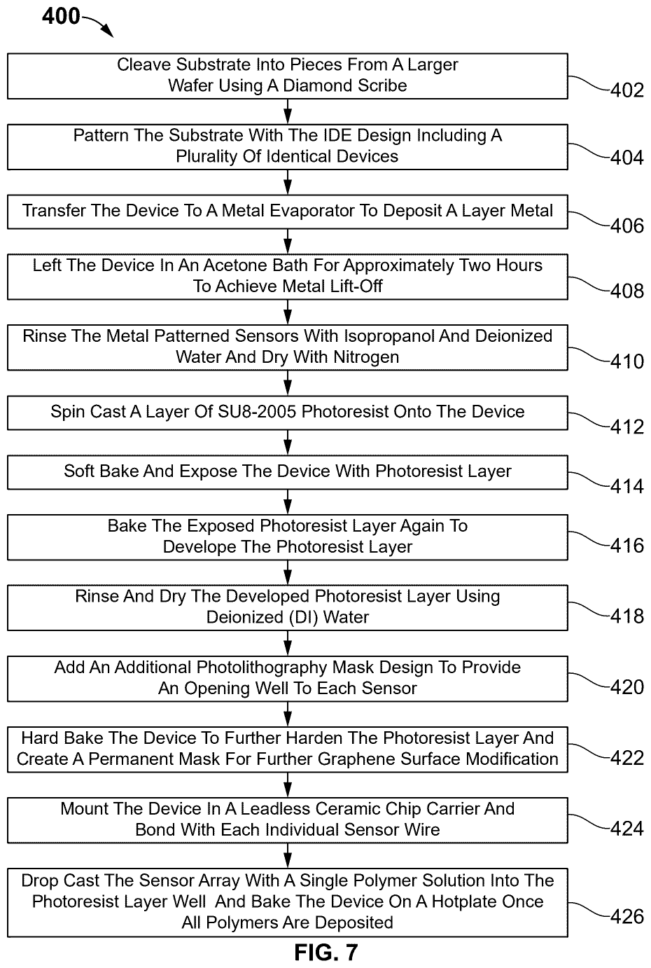

Referring to FIG. 7, a method 400 of fabrication of the sensor module 100, according to one embodiment of the present invention. In one embodiment, the sensor module 100 comprises the Si/SiO₂ substrate with monolayer graphene 24-element Ti/Au interdigitated electrodes (IDE), and a final SU8 layer with openings for each individual sensor 106 and its respective bonding pads. The Si/SiO₂/graphene substrate has a dimension of about 1.3 cm×1.3 cm.

The method 400 comprises the following steps. At step 402, the Si/SiO₂/graphene substrate is cleaved into pieces from a larger wafer. The SiO₂/graphene substrate is cleaved into approximately 1.3 cm×1.3 cm pieces from the large 4″ wafer using a diamond scribe. At step 404, the graphene substrate is patterned using a conventional lift-off photo-lithography. In one embodiment, the graphene substrate is patterned with the IDE design which consists of 24 identical devices. Each device comprises 12 electrode fingers with length, width, and separation of about 750 μm, 50 μm, and 50 μm, respectively, producing a total sensor area of about 0.44 mm².

At step 406, the device is then transferred to a metal evaporator. In the metal evaporator, a layer of Ti/Au is deposited once an appropriate base pressure is achieved. The Ti/Au layer has a dimension of about 25 nm/300 nm. At step 408, following metal deposition, the device is left in an acetone bath for approximately two hours to achieve metal lift-off. At step 410, the metal patterned sensors are then rinsed with isopropanol and deionized water and dried with nitrogen. At step 412, once the metal deposition process is complete, a layer of SU8-2005 photoresist is spun onto the device at a thickness of approximately 5 μm. The device with photoresist layer is rotated at a high speed. Initially, the device with photoresist layer is rotated at a speed of about 500 rpm for about 4 seconds. Then it is rotated at a speed of about 3000 rpm for about 30 seconds. The rotation spreads the photoresist evenly over the surface of the device.

At step 414, the device with photoresist is soft-baked at a temperature of about 110 °C for about 60 seconds. The photoresist layer is then exposed for about 4 seconds. At step 416, the exposed photoresist layer is again baked at a temperature of about 110 °C for about 60 seconds. The photoresist layer is developed approximately in 35 seconds. At step 418, the developed photoresist layer is rinsed using deionized (DI) water and dried. At step 420, an additional photo-lithography mask design is added to provide an opening well to each sensor with length, width, and depth of 1300 μm, 1200 μm, and 5 μm, respectively. At step 422, the device is then hard-baked at 150 °C for about 5 minutes to further harden the photoresist layer and create a permanent mask for further graphene surface modification. At step 424, the device is mounted in a 68-pin leadless ceramic chip carrier and each individual sensor wire is bonded.

In one embodiment, polymer coating solutions are created at a concentration of 20 mg/mL from 23 commercially available polymers. At step 426, following wire bonding, the sensor array is completed by drop casting approximately 0.4 μL of a single polymer solution into the photoresist layer well with a Hamilton microliter syringe, and baking the device on a hotplate at 70 °C for about 5 minutes once all polymers are deposited. Each well created by the photoresist layer or sensing layer 104 allows for the polymer solution to be contained in each individual sensor, and helps to prevent accidental mixing of the solutions. For the given concentration and deposition volume, approximately 8 μg of the polymer is estimated to be left on the sensor surface after solvent evaporation.

In one embodiment, the sensor arrays 100 with multiple specifically designed sensors 106 have been successfully created. The e-nose, being inspired by the biological functions of the olfactory system, is designed to contain an array of broadly responding (i.e., cross-reactive) sensors. The array elements respond to a broad number of compounds, with specificity being achieved by relationships and patterns across the array. In one embodiment, the sensor data or sensory information extracted from each sensor is encoded in features. The main attraction of this type of sensor array design is that if the sensor array is designed with enough dimensionality, it would be capable of detecting and classifying new, previously unforeseen compounds.

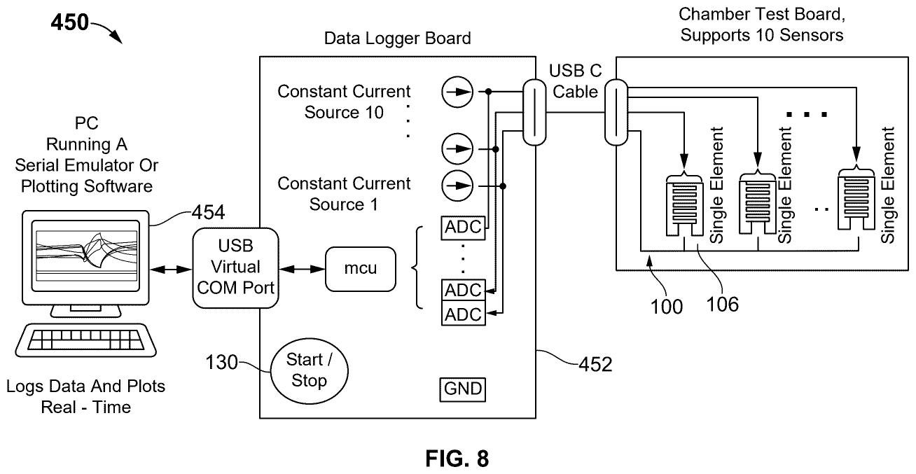

FIG. 8 exemplarily illustrates an environment 450 of a graphene-based chemical sensor system, according to an embodiment of the present invention. The system comprises at least one sensor module 100, a data logger module 452 and a computing device 454. The data logger module 452 is in communication with the sensor module 100. The sensor module 100, on presenting chemical compounds on the sensing surfaces and applying constant bias across the electrodes 108, is configured to create a sensor data with cross-reactive response characteristics due to temporary time-dependent change in resistance of the graphene. The data logger module 452 is configured to collect sensor data from the sensor module 100.

The computing device 454 is in communication with the data logger module 452. The computing device 454 comprises an artificial intelligence module. The computing device 454 further comprises machine learning module. The artificial intelligence module is trained to classify and identify a predefined set of chemical compounds. The computing device 454 is further configured to analyze the sensor data to identify chemical compounds by measuring a time-dependent change in resistance of the sensing surfaces. The computing device 454 is further configured to retrain the artificial intelligence module to retrain a set of new chemical compounds and add the new chemical compounds to the pre-defined set of chemical compounds. The analysis of the sensor data involves preprocessing the sensor data, extracting features from the sensor data, reducing the dimensionality of the established features, if necessary, and classifying chemical compounds. The computing device 454 is further configured to plot the cross-reactive response characteristics in real time. The raw responses could also be logged by connecting the data logger module 452 to the computing device 454, for example, a personnel computer (PC) via USB and using a serial emulator. The computing device 454 is further configured to control or change drive current based on a sensor resistivity measurement. The data logger module 452 comprises start and stop buttons 130 to manually control logging. The computing device 454 is further enable to control loggings by providing software commands to the data logger module 452.

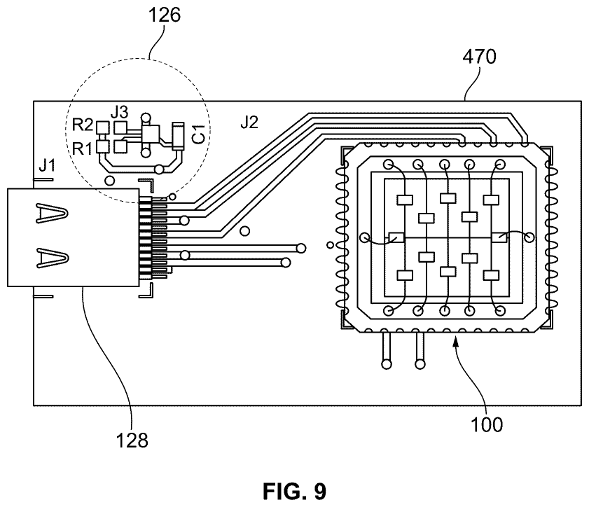

FIG. 9 exemplarily illustrates a chamber board 470 with a sensor module 100, according to an embodiment of the present invention. Referring to FIG. 1 and FIG. 9, the sensor module 100 is fixed to a leadless chip carrier 122. Further, the sensor module 100 is bonded 124 to the leadless chip carrier 122. The chamber board 470 further comprises a temperature sensor and a humidity sensor 126. Further, the chamber board 470 comprises at least one port 128 to connect the sensor module 100 to the data logger module 452. In one embodiment, the port 128 is a USB-C port for communication to the data logger module 452.

In one embodiment, the sensor array 100 is trainable and adaptable in the sense that the underlying machine learning (ML) and artificial intelligence (AI) modules could be re-trained to accommodate new compounds without the need for changing the sensor or hardware itself. The sensor module 100 provides ideal data for use in classification-based machine learning algorithms. The algorithms used are mature and understood and are capable of being re-trained to accommodate additional targets. Also, the algorithms are expandable to accommodate additional feature data without compromising accuracy. In one embodiment, the sensor module 100 is built upon modern data processing and architecture designs. The modern data processing and architecture designs involves: pre-processing of individual sensor data, extracting important features, for example, transient kinetic information, and training a machine learning algorithm to classify specific compounds. All the above steps of modern data processing are performed in a completely automated manner.

In one embodiment, the sensor module 100 could be dropped into a remote location. The sensor module 100 may either operate offline where it collects data onboard or in a networked configuration where it can communicate with other sensors. In one embodiment, the graphene sensor array 100 is desired for the sensor to be deployed to a remote location where there is no connectivity and it only relays its information on occasion or once, when the collection objective is complete. In one embodiment, multiple sensors may be networked together enabling additional geospatial data based on the individual sensor's location and their relative proximity. This additional data may provide knowledge about location, movement, and intent of the compounds being sensed.

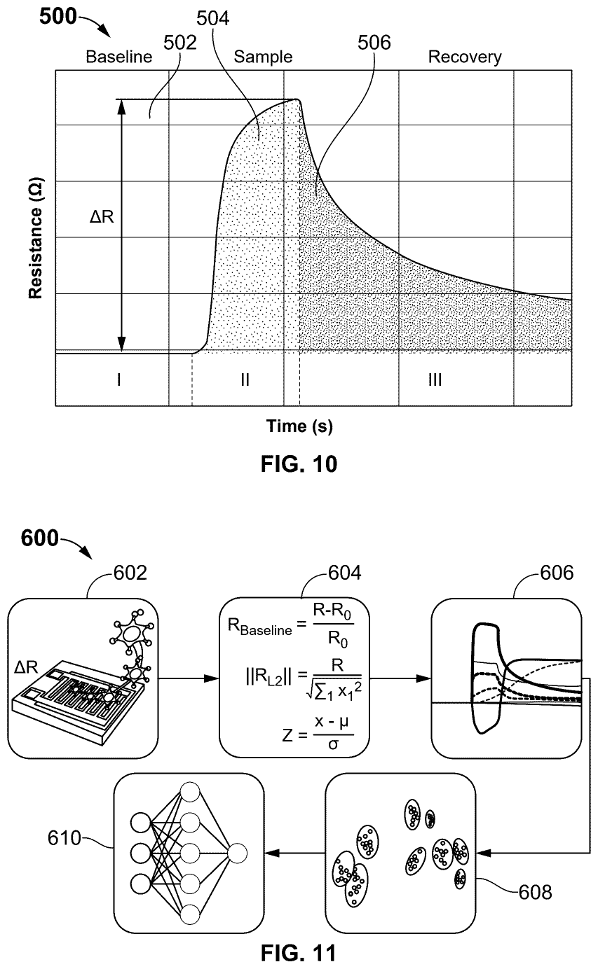

FIG. 10 illustrates a graph 500 illustrating a typical sensor response, according to one embodiment of the present invention. The graph 500 illustrates the various components of the typical sensor response. The sensor response is calculated based on the change in sensor resistance (Ω) with respect to time(s). The sensor response is labeled in three distinct regions. The three distinct regions include baseline region 502, sample region 504, and recovery region 506. The sensor response remains stable in the baseline region 502 and reaches a peak point in the sample region 504. The sensor response gradually decreases in the recovery region 506.

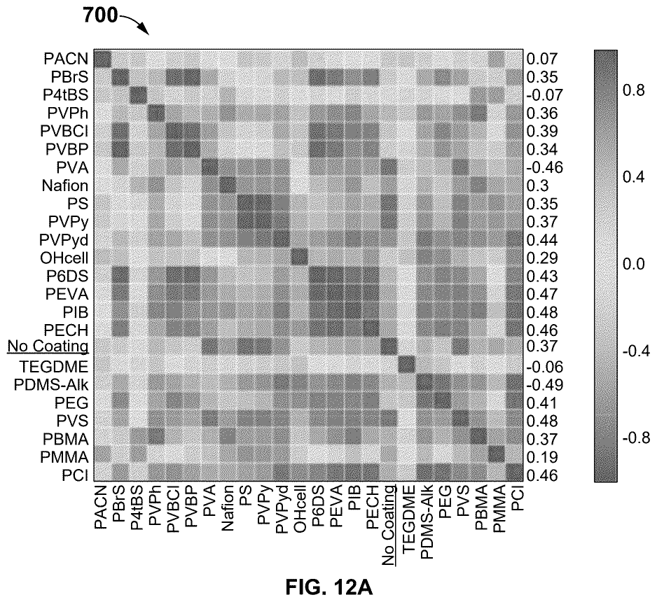

Referring to FIG. 11, a flowchart 600 of a method for classifying target compounds, according to one embodiment of the present invention. The flowchart 600 comprises the following steps. At step 602, a plurality of sensor data is obtained from each sensor in the graphene sensor array. At step 604, the sensor data is preprocessed to calculate sensor response. The sensor response is calculated using the change in resistance of the sensor.

$${R}_{Baseline} = \frac{R - {R}_{0}}{{R}_{0}}$$

$$‖ {R}_{L 2} ‖ = \frac{R}{\sqrt{{\sum }_{1} {x}_{1}^{2}}}$$

$$Z = \frac{x - μ}{σ}$$

At step 606, feature extraction is performed from the sensor data. The extracted features are shown in a graphical representation. At step 608, dimensionality reduction of the established features is performed. The dimension of each target compound is identified for classification. At step 610, the target compounds are classified. In one embodiment, the sensor array is adaptable in the sense that the underlying machine learning (ML) and artificial intelligence (AI) algorithms can be re-trained to accommodate new compounds without the need for changing the sensor or hardware itself.

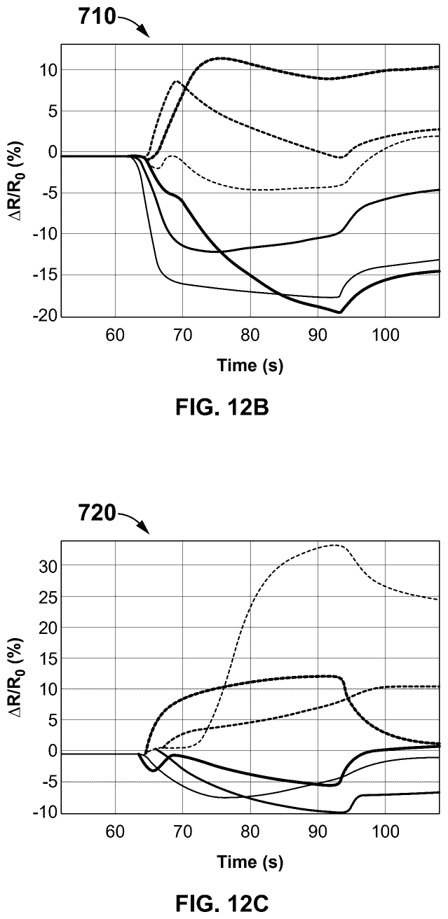

Referring to FIG. 12A, a graph 700 illustrating the change in resistance of various sensors, according to one embodiment of the present invention. The graph 700 shows the change in resistance (Ω) of various sensors with respect to time(s). The graph 700 shows the change in resistance of 24 graphene sensors with various surface modifications to 4 different compounds. The compounds may be acetone, acetonitrile, methanol, and tetrahydrofuran. FIGS. 11B-11C exemplarily illustrate the time-series towards 2 compounds for only a subset of the graphene sensors in different graphs (710 and 720), respectively. The graphs (710 and 720) reveal the rich transient information present in the sensor responses.

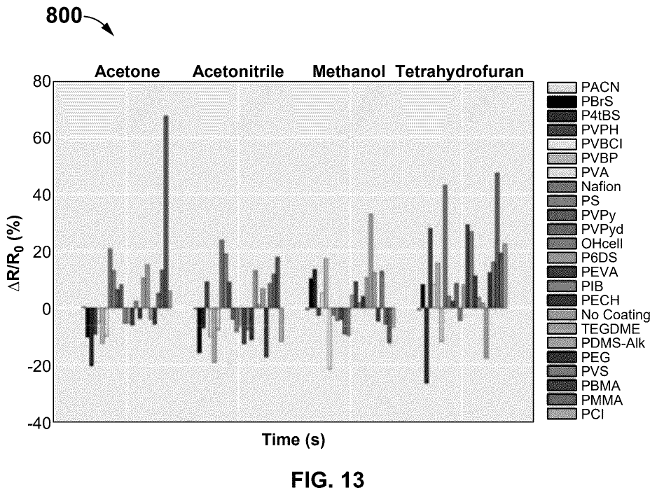

Referring to FIG. 13, a correlation heatmap 800 between any two modified sensors, according to one embodiment of the present invention. The legend to the right indicates the level of correlation, with different colors. For example, the correlation is indicated using dark red towards dark blue. The dark red indicates a strong positive correlation, while dark blue indicates a strong negative correlation. Using this same data in a hierarchical clustering analysis reveals grouping between modified sensors based on their relative dissimilarity to each other.

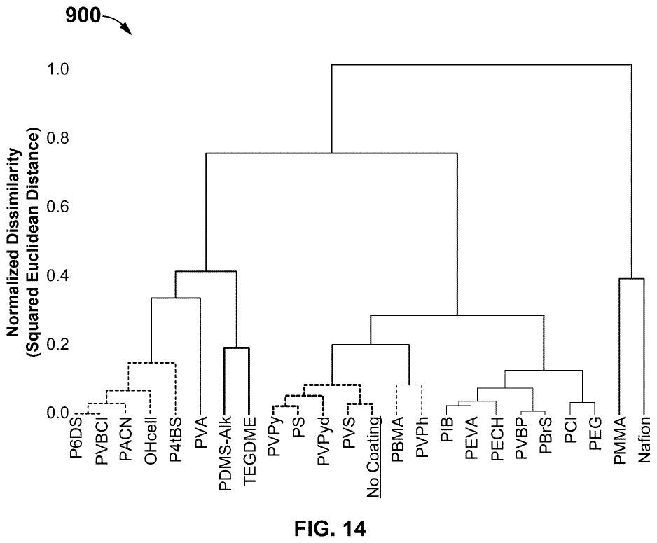

Referring to FIG. 14, specific algorithmic steps 900 to determine an optimal set of polymer coatings, according to one embodiment of the present invention. The polymer coatings are used to produce the most diverse and broadly responsive sensor array output. The broad, cross-reactive response of the sensor array lends itself to machine learning (ML) and artificial intelligence (AI) methods. The combination of the sensor array and sensor data processing steps create an adaptable device that is capable of sensing new and unforeseen compounds without changing the hardware and simply applying a software update.

According to the present invention, the sensor module 100 is capable of detecting and classifying a broad range of chemicals and compounds such as dozens of VOCs, chemical warfare simulants, and explosive material simulants. The sensor module 100 is also used to detect and classify complex odors found in panels of scotch whiskies and essential oils. In one embodiment, the sensor module 100 is adaptable to new compounds by use of training and machine learning. In one embodiment, the sensor module 100 may be deployed in a variety of form factors and environments. The sensor module 100 is cost effective, which may cost less than $200.

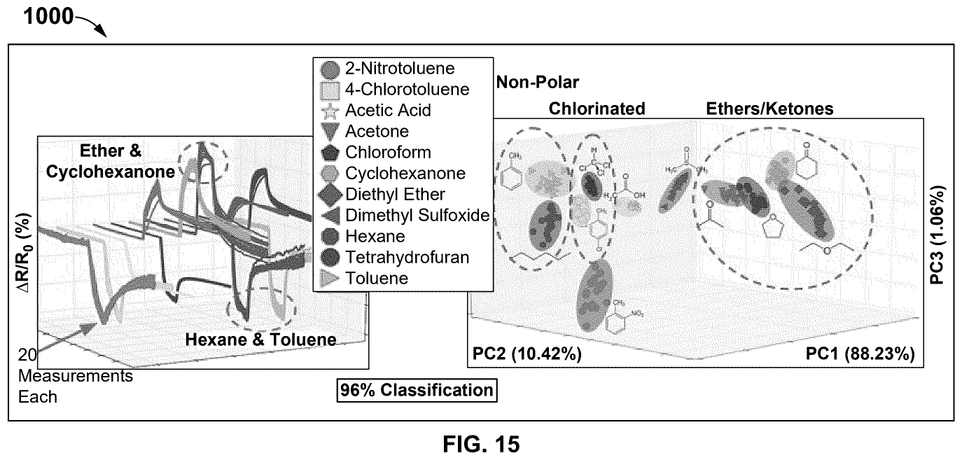

Referring to FIG. 15, a graph 1000 illustrating a diverse sensor response against a broad range of chemicals and compounds, according to one embodiment of the present invention. The graph 1000 shows the resultant response of the sensor module 100 that has been tested against a high classification of a number of VOCs, such as dozens of VOCs. The graph 1000 provides 96% of classification of VOCs.

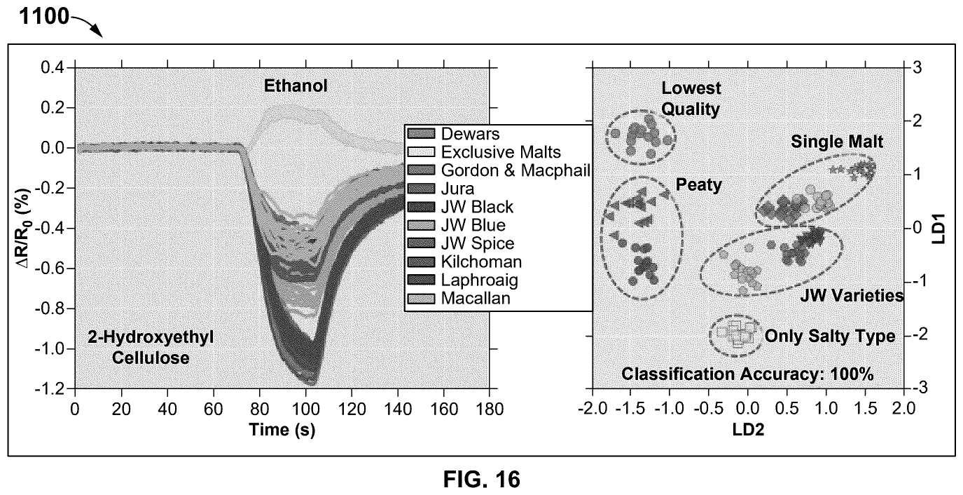

Referring to FIG. 16, a graph 1100 illustrating a classification accuracy of the complex odor from multiple scotch whiskies achieved by a single graphene sensor, according to one embodiment of the present invention. The graph 1100 shows 100% of classification accuracy of chemical warfare simulants and explosive material simulants. The graph 1100 also shows classification accuracy for the complex odors found in panels of scotch whiskies.

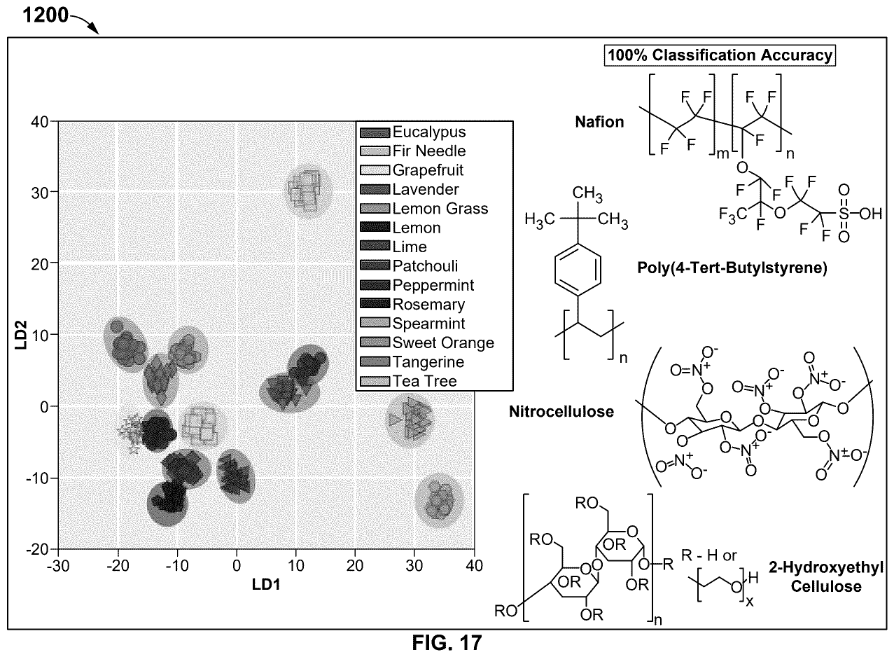

Referring to FIG. 17, a graph 1200 illustrating a classification accuracy of the complex odor from multiple essential oils achieved by four modified graphene sensors, according to one embodiment of the present invention. The graph 1200 shows 100% classification accuracy of the complex odor from multiple essential oils achieved by four modified graphene sensors.

The commercially available polymers are used to modify the sensor module 100 to produce the diverse response in the sensor output that has proved adaptable to new and unrelated chemicals, compounds, and complex odors, which requires a change to the training algorithm, but not the hardware itself. In practice, this process could be also carried out in a laboratory setting and resulting algorithm update applied to the device remotely. From a deployability standpoint, the sensor module 100 benefits from modern manufacturing and data processing standards. The sensor module 100 is compatible with standard semiconductor processing facilities and techniques and benefits from the maturely developed miniaturization techniques, which reduce size, weight, and power consumption. The processing of the sensor data could be carried out either on-board or remotely in a cloud computing environment such as AWS.

Applications of the sensor module 100 of the present invention includes, but not limited to the following. The sensor module 100 has the ability to adapt to detect new compounds, which lends itself to be operated in a variety of complex environments. In addition, the sensor module 100 may be used to provide situational awareness using not only the detection ability of each individual sensor, but also their collective geospatial relationship, which may provide knowledge about location, movement, and intent of the compounds being sensed. The sensor module 100 can self-destruct or burn up after set time or if picked up by someone (other than US forces).

Advantageously, the sensor module 100 of the present invention provides increased sensitivity and selectivity. The performance of the sensor module 100 is evaluated against single compounds and complex odors. The sensor array also operates at room temperature and does not require regenerative techniques such as heat or UV light, which are commonly found in other chemical sensing designs. These results demonstrate for the first time, a cross-reactive graphene chemical sensor array, applicable to compact, low-cost, low-power, and adaptable sensing needs for electronic nose systems.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A graphene-based chemical sensor system, comprising:
at least one sensor module comprising:
a substrate, wherein the substrate comprises graphene,
a plurality of pairs of electrodes disposed over the substrate, and
a sensing layer disposed over each pair of electrodes on the substrate to form an array of sensing surfaces, wherein the sensing layer is an integrated surface functionalization mask having an opening well for each sensing surface, wherein each opening well comprises a different polymer material,
wherein the sensor module, on presenting chemical compounds on the sensing surfaces and applying constant bias across the electrodes, is configured to create a sensor data with cross-reactive response characteristics due to temporary time-dependent change in resistance of the graphene;
a data logger module in communication with the sensor module configured to collect sensor data from the sensor module, and
a computing device in communication with the data logger module, wherein the computing device comprises an artificial intelligence module, wherein the computing device is configured to analyze the sensor data to identify chemical compounds by measuring a time-dependent change in resistance of the sensing surfaces.

2. The system of claim 1, wherein the artificial intelligence module is configured to classify and identify a predefined set of chemical compounds.

3. The system of claim 2, wherein the computing device is configured to re-train the artificial intelligence module to identify a set of new chemical compounds and add the new chemical compounds to the predefined set of chemical compounds.

4. The system of claim 1, wherein the substrate further comprises silicon (Si) and thermally grown silicon dioxide (SiO₂).

5. The system of claim 1, wherein the electrode is a titanium/gold (Ti/Au) interdigitated electrode.

6. The system of claim 1, wherein the sensing layer is an epoxy-based negative photoresist.

7. The system of claim 1, wherein the sensing layer is a SU8 layer.

8. The system of claim 1, wherein the sensor module further comprises at least one humidity sensor and at least one temperature sensor.

9. The system of claim 1, wherein the analysis of the sensor data involves preprocessing the sensor data, extracting features from the sensor data, reducing the dimensionality of the established features, if necessary, and classifying chemical compounds.

10. The system of claim 1, wherein the surface functionalization mask enables simultaneous deposition of various polymer materials to the sensing surfaces using microarray printing.

11. The system of claim 1, wherein the data logger module comprises a start and stop button to manually control logging of the sensor data.

12. The system of claim 1, wherein the computing device further enables to send commands to the data logger module to control logging of the sensor data.

13. The system of claim 1, wherein the computing device is further configured to plot the cross-reactive response characteristics and monitor sensor module in real time.

14. A graphene-based chemical sensor system, comprising:
at least one sensor module comprising:
a substrate, wherein the substrate comprises graphene,
a plurality of pairs of electrodes disposed over the substrate, and
a sensing layer disposed over each pair of electrodes on the substrate to form an array of sensing surfaces, wherein the sensing layer is an integrated surface functionalization mask having an opening well for each sensing surface, wherein each opening well comprises a different polymer material.

15. The system of claim 14, wherein the substrate further comprises silicon (Si) and thermally grown silicon dioxide (SiO₂).

16. The system of claim 14, wherein the electrode is a titanium/gold (Ti/Au) interdigitated electrode.

17. The system of claim 14, wherein the sensing layer is an epoxy-based negative photoresist.

18. The system of claim 14, wherein the sensing layer is a SU8 layer.
